(19) United States
(12) Patent Application Publication
(10) Pub. No.: US 2015/0274701 A1
(43) Pub. Date: Oct. 1, 2015

(54) NEW AZIDES, METHOD FOR PRODUCING SAME AND APPLICATIONS THEREOF

(71) Applicant: COMMISSARIAT A L'ENERGIE ATOMIQUE ET AUX ENERGIES ALTERNATIVES, Paris (FR)

(72) Inventors: Frederic Taran, Gif Sur Yvette (FR); Manon Chaumontet, Issy Les Moulineaux (FR); Valentine Bevilacqua, Paris (FR)

(21) Appl. No.: 14/432,526

(22) PCT Filed: Oct. 8, 2013
(86) PCT No.: PCT/FR2013/052384

§ 371 Date: Mar. 31, 2015

(30) Foreign Application Priority Data
Oct. 8, 2012 (FR) 12 59571

(51) Int. Cl. C07D 403/12; G01N 33/94

(52) U.S. Cl. CPC C07D 403/12; G01N 33/94

(57) ABSTRACT

Azides of formula (I) in which V and W represent, independent of each other, a heterocycle comprising a copper-complexing nitrogen or sulphur heteroatom, said heterocycle being saturated or unsaturated, X and Y represent, independent of each other, a spacer moiety; Z represents a spacer moiety, F and F2 represent, independent of each other, a hydrogen atom or a functional moiety allowing the coupling of the azide of formula I with a chemical molecule, a biomolecule, a nanoparticle or a polymer, it being understood that at least one of F1 and F2 represents a functional moiety, methods of production and uses of same.

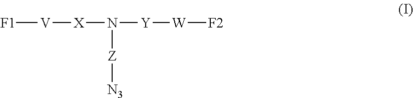

The present invention relates to molecules which have in their structure an azide function (N₃) and chemical groups which allow the complexing of a copper atom, to processes for the production thereof and to applications thereof.

Azide/alkyne click reactions are among the chemical reactions most cited in the literature; there are in particular thousands of scientific articles and more than 400 patents relating to the application to said reaction. This reaction requires copper Cu¹⁺ which acts as a catalyst. However, for a good number of applications, the experimental conditions (in particular conditions of low reagent concentrations) in practice require the use of excess copper which proves to be detrimental when working with cells, biomolecules or certain nanoparticles. Copper is in fact toxic to cells and can degrade or denature proteins or cause certain nanoparticles to lose properties (loss of quantum dot fluorescence for example).

Three solutions to this problem are described in the literature:

The first consists in using cyclic alkynes, the reactivity of which is sufficient to dispense with a copper catalyst. These systems are in particular described by the team of Prof. Bertozzi (see publications 1-4). The main drawback of this technique is relatively slow kinetics, which can result in low coupling yields, in particular when two macromolecules must be coupled.

The second consists in using Cu¹⁺ ligands which allow an acceleration of the reaction. Since the reaction kinetics are increased, it is then possible to reduce the amount of copper (see publication 5). Despite the use of increasingly effective copper ligands, this technique again involves amounts of copper that are too high for many applications, in particular that involving cells or biomolecules and fragile nanoparticles.

The third consists in using azides which have a particular reactivity owing to their capacity to bind copper (see publication 6). However, this technique is limited to azides which have two nitrogen atoms binding the copper, including that of the azide function, as shown in the formula below.

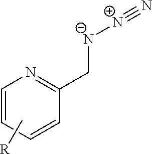

However, the corresponding copper complexes are therefore not stable, thereby limiting their field of application: the use of ⁶⁴Cu for example is impossible and intracellular or in vivo ligation is difficult to envision. The application of these compounds, which is ligation on the surface of cells, requires the addition of copper ligand. However, under these conditions, the ligation kinetics are limited.

It will therefore be desirable to have means allowing an azide/alkyne click reaction which does not require the use of an excess of copper.

It would also be desirable to have a reaction, the kinetics of which are sufficiently rapid and the biocompatibility of which is sufficiently high to maintain good effectiveness even under conditions of high dilution and in the presence of the molecular complexity of biological media.

In summary, it would therefore be desirable to have novel reagents capable of sequestering copper while at the same time conferring thereon an increased reactivity for the azide/alkyne click reaction.

After lengthy research, the applicant has developed a bifunctional azide having a copper ligand structure, which is capable of acting as both a reagent and a catalyst. It consequently allows a bimolecular azide/alkyne click reaction.

Consequently, a subject of the present application is an azide of formula I

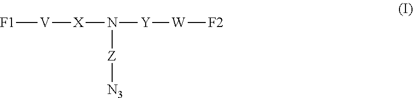

wherein:

V and W represent, independently of one another, a nitrogenous or sulfur-containing heterocycle comprising a copper-complexing heteroatom, said heterocycle being saturated or unsaturated,

X and Y bonded to the central nitrogen represent, independently of one another, a spacer group, in particular a group of structure —(CH₂)₂—O—(CH₂)—, —(CH₂)—O—(CH₂)₂—, —(CH₂)—O—(CH₂)—, —(CH₂)₂—S—(CH₂)—, —(CH₂)—S—(CH₂)₂—, —(CH₂)—S—(CH₂)—, —(CH₂)₂—NH—(CH₂)—, —(CH₂)—NH—(CH₂)₂—(CH₂)—NH—(CH₂)—, or —(CH₂)ₘ—, wherein n can take the values 1, 2, 3 or 4, preferably a —(CH₂)ₘ— group, wherein n can take the values 1, 2, 3 or 4;

Z represents a spacer group, in particular a group of structure —(CH₂)₂—O—(CH₂)—, —(CH₂)—O—(CH₂)₂—, —(CH₂)₂—S—(CH₂)—, —(CH₂)—S—(CH₂)₂—, —(CH₂)₂—NH—(CH₂)—(CH₂)—NH—(CH₂)₂—, or —(CH₂)ₙ—, wherein n can take the values 1, 2, 3 or 4, preferably a —(CH₂)ₙ— group, wherein n can take the values 1, 2, 3 or 4;

F1 and F2 represent, independently of one another, a hydrogen atom, or a functional group which allows the coupling of the azide of formula I to a chemical molecule, to a biomolecule, to a nanoparticle or to a polymer, it being understood that at least one of F1 and F2 represents a functional group. F1 and F2 can in addition represent, independently of one another, a tertiary alkylene group having at most 7 carbon atoms, preferably at most 5 carbon atoms, in particular a tert-butyl group.

When the heterocycles are nitrogenous heterocycles, they are advantageously pyridine, imidazole, pyrimidine, pyrizine, triazine, imidazole, azepane, oxazole, pyrrole, morpholine, isoxazole, imidazole, pyrazole, oxadiazole, triazole, tetrazole, benzimidazole, benzoxazole, pyridazine, quinoline, isoquinoline, quinoxaline, phthalazine, quinazoline, benzotriazine, 1,4,8,11-tetraazacyclotetradecane (cyclam) or 1,4,7,10-tetraazacyclododecane (cyclen). In this heterocycle, the copper-complexing heteroatom is preferably set apart from the central nitrogen of the azide of formula (I) by two to five atoms, preferably carbon atoms, in particular by two or three atoms, preferably carbon atoms.

When the heterocycles are sulfur-containing heterocycles, they are advantageously isothiazole, thiazoles, thiophene, thiadiazole, benzothiazole or thiadiazines.

The heterocycles are preferably chosen from nitrogenous heterocycles, advantageously pyridine, imidazole, pyrrole, triazole and benzimidazole.

As is well known to those skilled in the art, see for example Wikipedia, “A molecular spacer or more simply a spacer is, in the chemistry field, any variable part of a molecule providing a connection between two other parts of a molecule.” Such a part can vary without changing the functionality of the molecule in question.

X preferably represents a methylene, ethylene or propylene group, particularly a methylene or ethylene group, more particularly a methylene group. In another embodiment, X represents an ethylene or propylene group.

Y preferably represents a methylene, ethylene or propylene group, particularly a methylene or ethylene group, more particularly a methylene group. In another embodiment, Y represents an ethylene or propylene group.

Z preferably represents an ethylene, propylene or butylene group, preferably a propylene group.

At least one of F1 and F2 preferably represents, independently of the other, a tertiary alkylene group having at most 7 carbon atoms, preferably at most 5 carbon atoms, in particular a tert-butyl group, or a functional group which allows the coupling of the azide of formula (I) to a chemical molecule, a biomolecule, a nanoparticle or a polymer. These functional groups may vary to large extents, depending on the nature of the compound to which the azide will be coupled. F1 and F2 comprise for example an isocyanate or isothiocyanate group and preferably a carboxyl, amino, maleimide or thiol radical. These functional groups are indirectly grafted onto the heterocycles, for example by means of an alkylene group, in particular an alkylene group having from 1 to 6 carbon atoms, particularly from 2 to 5 carbon atoms.

For example, in order to couple an azide of formula (I) to an aminated chemical molecule, a carboxyl radical will for example be chosen, and, conversely, in order to couple an azide of formula (I) to a chemical molecule comprising a carboxyl radical, an amino radical will preferably be chosen. This carboxyl radical will also be of use, for example, in coupling to a biomolecule formed from amino acids or comprising amino acids.

The chemical molecule is preferably a fluorophore, a ligand, or an active ingredient for therapeutic or phytosanitary use. The term “ligand” is intended to mean a compound capable of binding to a receptor.

The biomolecule is preferably an antibody, a protein, a polysaccharide or a polynucleotide.

A nanoparticle is a synthetic particle, one dimension of which is less than 100 nm, and for example a carbon nanotube, a quantum dot or a micelle, and preferably a quantum dot.

The polymer is, for example, polystyrene, polyacrylamide or polypropylene, and preferably polyacrylamide.

Among the azides of formula I described above, the compounds of formula I wherein X and Y represent, independently of one another, a methylene, ethylene or propylene group, particularly a methylene or ethylene group, and F1, F2, V, W and Z have the meaning already indicated, are in particular selected.

The compounds of formula I above wherein Z represents a —(CH₂)ₙ— group wherein n has the value 2 or 3 and X, Y, F1, F2, V, W, and Z have the meaning already indicated, are particularly selected.

The compounds of formula I above wherein X and Y represent, independently of one another, an ethylene or propylene group, Z represents an ethylene or propylene group, and F1, F2, V and W have the meaning already indicated, are also particularly selected.

The compounds of formula I above wherein X and Y represent, independently of one another, a methylene or ethylene group, more particularly a methylene group, and at least one of F1 and F2 represents, independently of the other, a tertiary alkylene group having at most 7 carbon atoms, preferably at most 5 carbon atoms, in particular a tert-butyl group, or a functional group, are more particularly selected, this functional group preferably being chosen from the specific functional groups described above, i.e. isocyanate or isothiocyanate groups, a carboxyl, amino, maleimide or thiol radical, and preferably one of the last four functional groups mentioned. In addition, among them, preference is given to those for which Z represents a —(CH₂)ₙ— group, wherein n can take the values 1, 2, 3 or 4, in particular an ethyl, propylene or butylene radical.

Even more particularly selected, especially among the preferred compounds above, are the compounds of formula I above wherein V and W represent, independently of one another, pyridine, imidazole, pyrrole, benzimidazole and triazole, particularly the latter, X and Y represent, independently of one another, a methylene, ethylene or propylene group, particularly methylene or ethylene, Z represents an ethylene or propylene group, and F1 and F2 have the meaning already indicated.

Among the particularly preferred compounds of the invention, mention may furthermore be made of:
5,5′-(4,4′-(((3-azidopropyl)azanediyl)bis(methylene))bis(1H-1,2,3-triazole-4,1-diyl))dipentanoic acid,
5-(4-(((3-azidopropyl)((1-(tert-butyl)-1H-1,2,3-triazol-4-yl)methyl)amino)methyl)-1H-1,2,3-triazol-1-yl)pentanoic acid and
4,4′-(4,4′-(((3-azidopropyl)azanediyl)bis(methylene))bis(1H-1,2,3-triazol-4,1-diyl))bis(N-(3-aminopropyl)butanamide).

It should be noted that, in the present application, conventionally the indefinite article “a” should be considered to be a generic plural (meaning of “at least one” or else “one or more”), except when the context shows the contrary (1 or “a single”). Thus, for example, when it is said above that an azide of formula I is used, it is a question of the use of one or more azides of formula I.

The subject of the present application is also a process for preparing an azide of formula I above, wherein X and Y represent, independently of one another, a —(CH₂)ₘ— group, wherein m has the value 1, 2, 3 or 4, characterized in that an amino alcohol of formula II

OH—Z—NH₂ (II)

wherein Z has the meaning already indicated, is reacted with an aldehyde of formula III

GP₁—F₁—V—X′—CHO (III)

wherein F₁ and V have the meaning already indicated, GP₁ represents a group which protects an F1 function and X′ represents a —(CH₂)ₘ₋₁— group wherein m has the value already indicated,
so as to obtain a compound of formula IV

GP₁—F₁—V—X′—CH₂—NH—Z—OH (IV)

wherein GP₁, F₁, V, X′ and Z have the meaning already indicated,
which is subjected, with an aldehyde of formula V

GP₂—F₂—W—Y′—CHO (V)

wherein F₂ and W have the meaning already indicated, GP₂ represents a group which protects an F2 function and Y′ represents a —(CH₂)ₘ₋₁— group wherein m has the value already indicated,
to a reductive amination so as to obtain a compound of formula VI

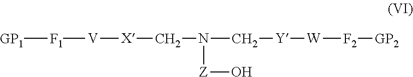

wherein GP₁, F₁, V, X′, Y′, W, F₂, GP₂ and Z have the meaning already indicated,
which is reacted with an alkali metal azide of formula VII

EN₃ (VII)

wherein E represents an alkali metal, preferably sodium,
so as to obtain a compound of formula VIII

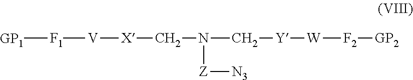

wherein GP₁, F₁, V, X′, Y′, W, F₂, GP₂ and Z have the meaning already indicated, from which the protective groups are removed so as to obtain the expected compound of formula IA

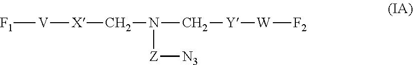

which is isolated if desired.

Under preferential conditions for carrying out the process described above,
the reductive amination reaction is carried out in the presence of a hydride;
to facilitate the reaction of the compound of formula VI with an alkali metal azide of formula VII, the alcohol function of the compound of formula VI is activated with a tosyl, mesyl or triflate group, preferably a mesyl group;
the removal of the protective groups GP₁ and GP₂ is preferably carried out using an aqueous sodium hydroxide solution in the case of a carboxylic acid F₁ or F₂ function protected in the form of an ester, and using trifluoroacetic acid in the case of an amine function protected in the form of a carbamate.

In summary, the above synthesis route consists in reacting an amino alcohol, the amine and alcohol functions of which are separated by a spacer Z, with a compound having an aldehyde function, a spacer X′ (corresponding to the lower homolog of the spacer X—loss of a chain member of the spacer X), a heterocycle V, and an F₁ function protected by a protective group GP₁ according to a reductive amination reaction. The resulting secondary amine can then undergo a second reductive amination reaction with a new compound having an aldehyde function, a spacer Y′ (corresponding to the lower homolog of the spacer Y), a heterocycle W, and an F₂ function protected by a protective group GP₂. In the case of the preparation of a symmetrical molecule (F₁═F₂, V═W and X═Y), the two reductive amination reactions can be carried out in a single step, for example using two equivalents of the aldehyde reagent.

The next step consists in activating the alcohol function with a mesyl group so as to facilitate the step of substitution with sodium azide (NaN₃) so as to form the corresponding azide.

A subject of the present application is also a process for preparing an azide of formula I above, wherein V and W represent a triazole, characterized in that a halogenated alkyne of formula Xa

≡—X—Br (Xa)

wherein X has the meaning already indicated, and Br represents a halogen atom, preferably a bromine atom,
is reacted with an amino alcohol of formula XI

OH—Z—NH₂ (XI)

wherein Z has the meaning already indicated,
so as to obtain a dialkyne of formula XII

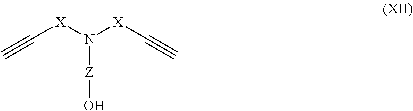

wherein X and Z have the meaning already indicated,
which is reacted with an azide of formula XIII

GP₁—F₁—N₃ (XIII)

wherein GP₁ and F₁ have the meaning already indicated, in the presence of a catalyst, preferably a copper-based catalyst,
so as to obtain by cycloaddition an alkyne of formula XIV,

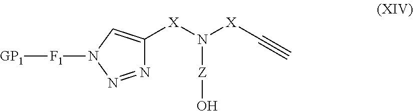

wherein GP₁, F₁, X and Z have the meaning already indicated,
which is reacted with an azide of formula XV

GP₂—F₂—N₃ (XV)

wherein GP₂ and F₂ have the meaning already indicated, in the presence of a catalyst, preferably a copper-based catalyst,
so as to obtain by cycloaddition a compound of formula XVI,

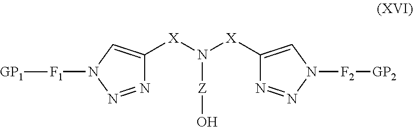

wherein GP₁, GP₂, F₁, F₂, X and Z have the meaning already indicated,
which is reacted with an alkali metal azide of formula VII

EN₃ (VII)

wherein E has the meaning already indicated,
so as to obtain a compound of formula XVII

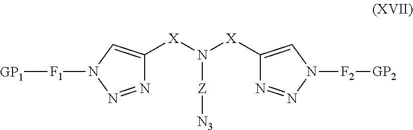

wherein GP₁, GP₂, F₁, F₂, X and Z have the meaning already indicated,
from which the protective groups are removed so as to obtain the expected compound of formula IB

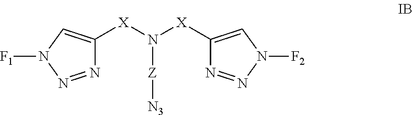

wherein F₁, F₂, X and Z have the meaning already indicated and which is isolated if desired.

Under preferential conditions for carrying out the process described above:
the reaction of a halogenated alkyne of formula Xa with an amino alcohol of formula XI is carried out in the presence of a base and preferably sodium carbonate or potassium carbonate, under moderate heating and preferably at 50° C.;
the reaction of the dialkyne of formula XII or the monoalkyne of formula XIV with an azide of formula XV is carried out in the presence of a copper-based catalyst and preferably in the presence of a reducing agent such as ascorbic acid;
the reaction of the compound of formula XVI with an alkali metal azide of formula VII is carried out in the presence of an activator of the alcohol function, such as a methanesulfonyl halide, for instance methanesulfonyl chloride, in a solvent such as triethylamine.

The azides of formula I which are the subject of the present invention have very advantageous properties. They have the capacity to complex copper and to react, via their azide function, with terminal alkyne functions according to the copper-catalyzed reaction for 1,3-dipolar cycloaddition of azides to alkynes, said reaction belonging to what is known as click chemistry.

The azides of formula I which are the subject of the present invention thus allow the covalent coupling (or ligation) between two identical or different entities (chemical molecules, nanoparticles, biomolecules, polymers, etc.), this being with great effectiveness even in complex media, such as biological media, for instance culture media and cell lysates or plasma. The ligation is also very chemoselective, which explains the fact that it can be carried out effectively even in complex media (cell media, blood, etc.), or even in vivo.

The azides of formula I which are the subject of the present invention are bifunctional since they act both as a reagent and as a catalyst. Consequently, the reaction thereof with the above entities becomes biomolecular (the entity and the azide), which is kinetically favorable compared with techniques which require the meeting of three partners.

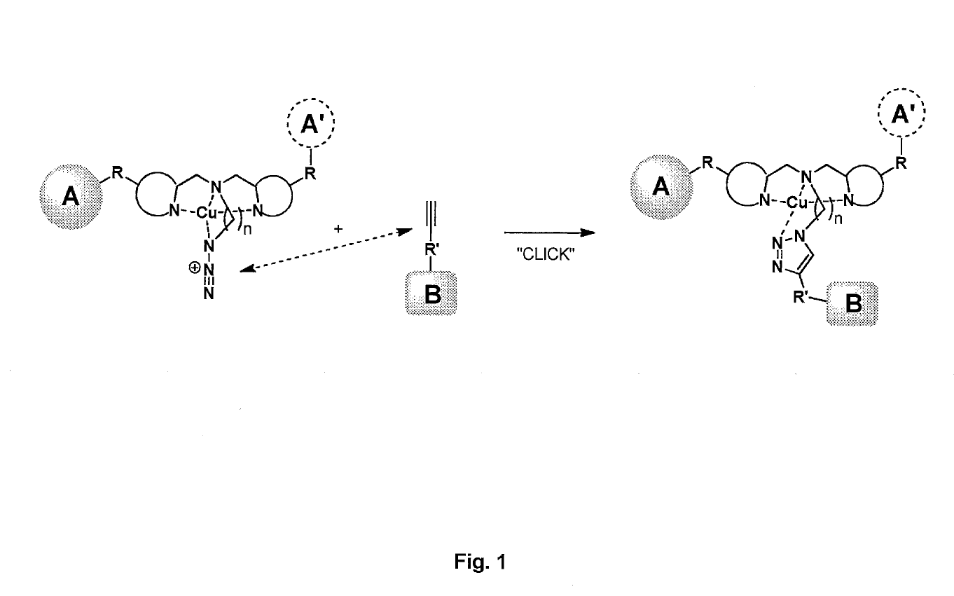

The reactivity of the azides of formula I which are a subject of the present invention for alkynes is such that the ligation reaction is extremely rapid; consequently, it is further effective even under conditions of high dilution, which is conventionally the case for bioconjugations, for example. By means of the F1 and F2 functional groups, the azides of formula I which are a subject of the present invention can be easily labeled with a fluorophore A′ and then covalently bonded to an antibody A for example. This antibody will then be able to react in the presence of copper with a nanoparticle B prefunctionalized with a terminal alkyne so as to produce the corresponding conjugate as illustrated by an example in FIG. 1.

These properties are illustrated hereinafter in the experimental section. They justify the use of the azides of formula I described above, in the preparation of molecule-nanoparticle conjugates, molecule-biomolecule conjugates, nanoparticle-biomolecule conjugates, biomolecule-polymer conjugates, etc.

They also justify the use of the azides of formula I, described above, in “fishing” techniques which make it possible to isolate and identify a protein target within complex biological mixtures or even in a cell, in particular human cell. The “fishing” techniques are conventionally carried out using the streptavidin/biotin technology (biotin grafted onto the molecule B and streptavidin immobilized on the support), of which the limitations due to the nonspecific interactions of biotin are known. The use of the azides of the present invention does not have such a limitation.

The “fishing” technique of the present invention can be implemented on whole cells, in contrast with the techniques where it is necessary to apply the technique to a cell lysate.

They also justify the use of the azides of formula I, described above, in imaging with ⁶⁴Cu complexes. This is because the copper remains bonded once the ligation reaction has been carried out.

The stability of the copper complex obtained with the azides of the formula I of the invention makes it possible to retain a high efficiency of the ligation reaction since the latter is carried out with a yield close to 100%, compared with less than 20% with the solutions of the prior art.

Consequently, a subject of the present application is also the use of the azides of formula I described above, in particular in
the preparation of bioconjugates or of nanoconjugates,
the isolation or the identification of a target, in particular a protein target, particularly within a complex biological mixture, for instance cell lysates, cell cultures or plasma,
imaging, in particular with ⁶⁴Cu complexes.

For use thereof in the preparation of bioconjugates or of nanoconjugates, the process may in particular comprise the following steps:
1) An azide of formula I is grafted onto a biomolecule or onto a nanoparticle.
2) An alkyne is grafted onto a molecule, a biomolecule or a nanoparticle.
3) The two grafted azide and alkyne partners are brought into contact in the presence of cuprous ions, as a result of which said partners are covalently bonded to one another.

For use thereof in the isolation or the identification of a target, in particular a protein target, particularly within a complex biological mixture, for instance cell lysate, the process may in particular comprise the following steps:
1) an azide of formula I is grafted onto a support, for example comprising beads, onto the walls of a container or onto a plate,
2) a medium containing a molecule B which binds to the target, in particular protein target, and which comprises a terminal alkyne function is brought into contact with this grafted support in the presence of cuprous ions, as a result of which said compound is attached to the support,
3) the target, for example protein target, thus attached to the support is digested with restriction enzymes such as trypsin and the analysis of the peptides thus generated allows the identification of said target.

For use thereof in imaging, in particular with ⁶⁴Cu complexes, the process may in particular comprise the following steps:
1) an alkyne is grafted onto a biomolecule or a nanoparticle,
2) a cuprous ⁶⁴Cu/azide of formula I complex is prepared,
3) the two partners are brought into contact, as a result of which said partners are covalently bonded to one another.

As will be seen hereinafter in the experimental section, the particular structure of the azides of the present invention allows an extremely rapid click reaction.

Consequently, a subject of the present application is also, as novel industrial products, of use in particular for the preparation of the azides of formula I above, the azides of formula XX

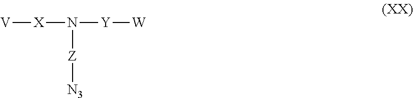

wherein X, Y, V, W and Z have the meaning already indicated for the azides of formula I.

The preferential conditions for using the azides of formula I described above also apply to the other subjects of the invention targeted above, in particular to the processes for producing same, to the uses thereof and to the azides of formula XX.

The examples and experiments which follow illustrate the present application, and the invention will be understood more clearly if reference is made to the appended drawings in which

FIG. 1 represents a first type of application of the azides of formula I of the present invention, to the preparation of conjugates. A and B may be molecules, biomolecules, nanoparticles or polymers. A′ may be a molecule such as, for example, a fluorophore;

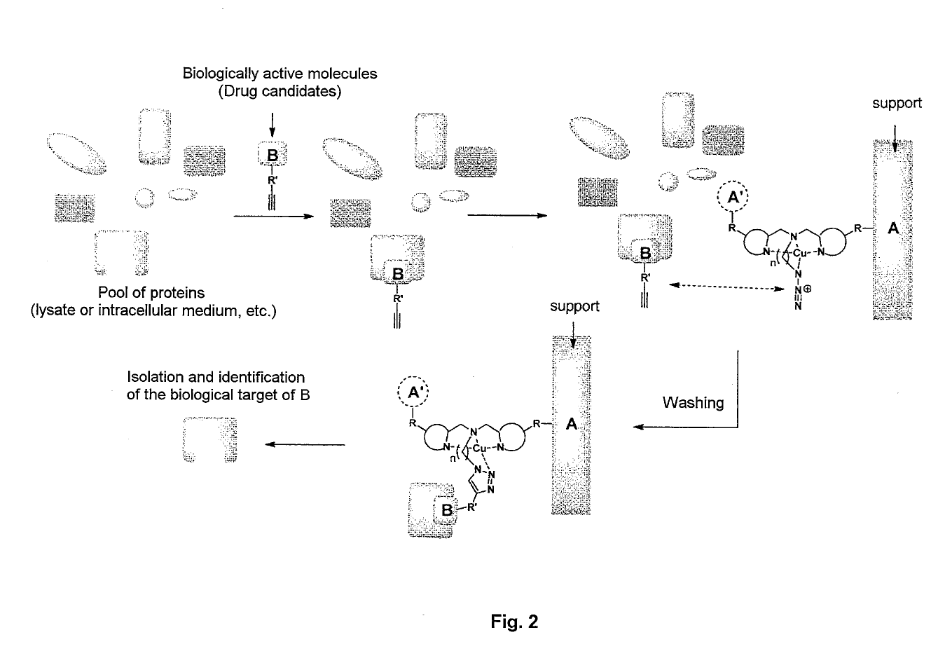

FIG. 2 illustrates the principle of an example of improving fishing techniques;

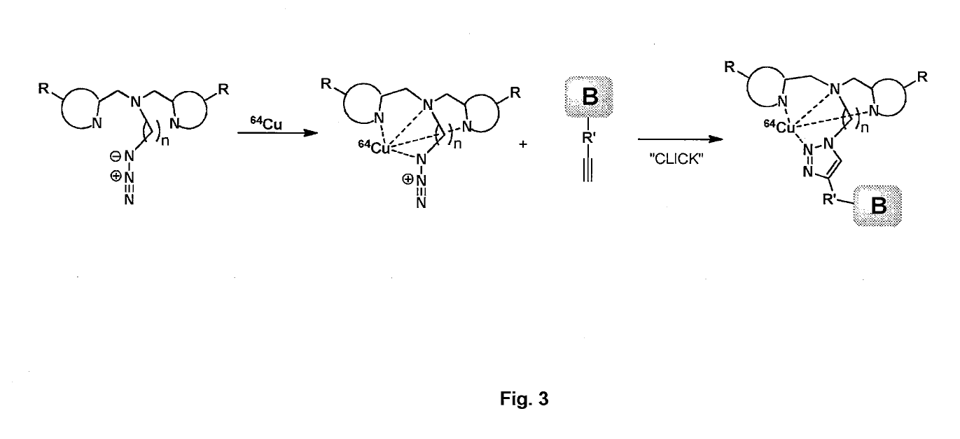

FIG. 3 illustrates the principle of an example of development of “clickable” radioactive complexes;

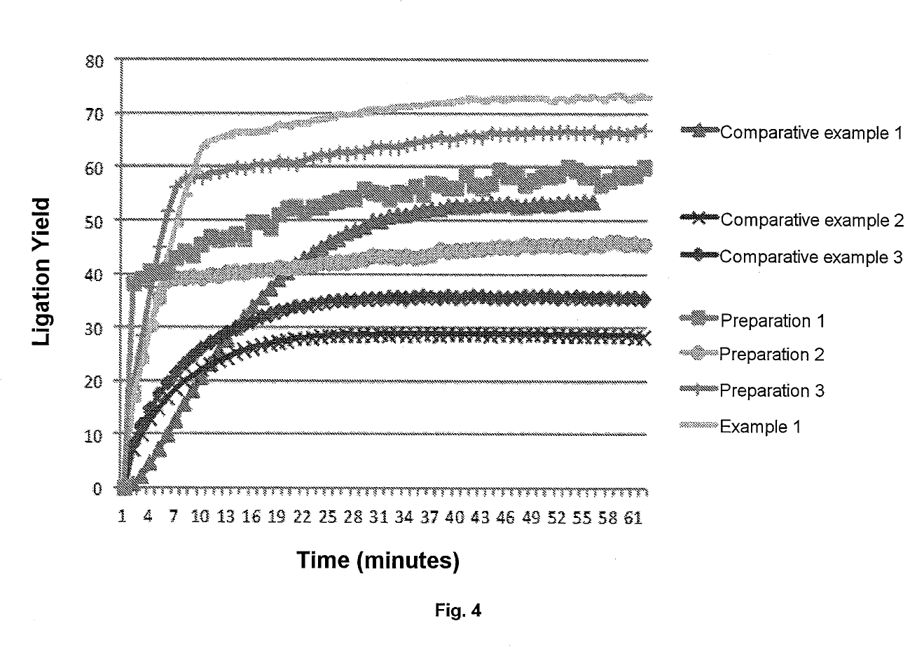

FIG. 4 is a graph representing the ligation yield expressed as percentage as a function of time expressed in minutes, obtained with azides of the invention and obtained with azides of the prior art, in a single medium (buffered aqueous solution);

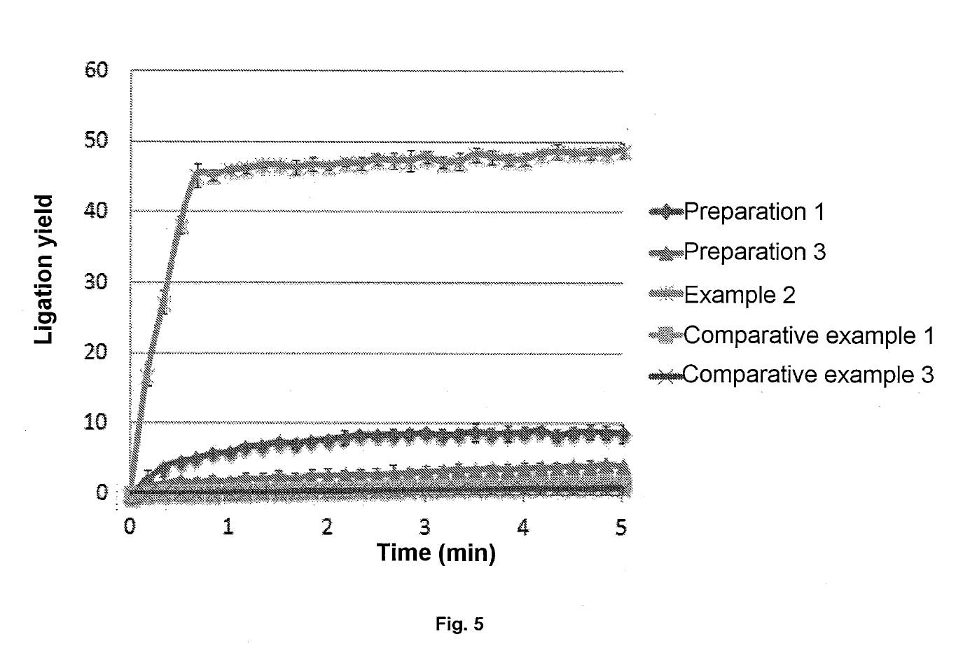

FIG. 5 is a graph representing the ligation yield expressed as percentage as a function of time expressed in minutes, obtained with azides of the invention and obtained with azides of the prior art, in a complex medium;

FIG. 6 is a graph representing the click reaction kinetics compared between various chelating azides in a phosphate buffer medium;

FIG. 7 is a graph representing the click reaction kinetics compared between various chelating azides in a cell lysate;

FIG. 8 represents confocal microscopy images of HuH-7 cells.

### PREPARATION 1

### 3-Azido-N,N-bis((1-methyl-1H-benzo[d]imidazol-2-yl)methyl)propan-1-amine (azide A1)

### Stage 1: 3-(bis((1-methyl-1H-benzo[d]imidazol-2-yl)methyl)amino)propan-1-ol

3-Aminopropanol (174 μl, 2.3 mmol) and acetic acid (260 μl, 4.6 mmol) are added to a solution of methyl-2-formyl benzimidazole (730 mg, 4.6 mmol) in THF anhydride (25 ml). The reaction mixture is then stirred for 48 h at ambient temperature under N₂ atmosphere. After concentration of the reaction crude under vacuum, the residue is taken up with CH₂Cl₂ and then washed with a saturated sodium bicarbonate solution. The aqueous phase is extracted twice with dichloromethane and then the organic phases are pooled, dried over MgSO₄ and concentrated. Finally, the reaction crude is purified by silica column flash chromatography (9/1 EtOAc/MeOH) to give the expected product in the form of a colorless oil (245 mg, 0.68 mmol, 30% yield).

¹H NMR (400 MHz, Chloroform-d) δ=7.70 (m, 2H), 7.24-7.19 (m, 6H), 3.96 (s, 4H), 3.82 (t, J=5.8 Hz, 2H), 3.67 (s, 6H), 2.92 (t, J=5.8 Hz, 2H), 1.84 (quin, J=5.8 Hz, 2H) ppm;

¹³C NMR (101 MHz, Chloroform-d) δ=151.5 (2C), 141.7 (2C), 135.9 (2C), 122.9 (2C), 122.3 (2C), 119.4 (2C), 109.3 (2C), 60.8, 53.5, 51.4 (2C), 30.1 (2C), 29.5 ppm;

IR (NaCl pellets): 3287, 2938, 1478, 1402, 1333, 174 cm⁻¹.

### Stage 2: 3-azido-N,N-bis((1-methyl-1H-benzo[d]imidazol-2-yl)methyl)propan-1-amine

Triethylamine (106 μl, 0.76 mmol) and mesyl chloride (59 μl, 0.76 mmol) are added to a solution of previously obtained alcohol (230 mg, 0.63 mmol) in anhydrous DMF and then the reaction medium is stirred at ambient temperature under an N₂ atmosphere. After disappearance of the starting alcohol (TLC control), sodium azide (205 mg, 3.2 mmol) is added to the reaction mixture. After 12 h of stirring, the reaction crude is concentrated under vacuum, and directly purified by silica column flash chromatography (9/1 EtOAc/MeOH) to give the expected product in the form of a white amorphous solid (172 mg, 0.44 mmol, 70% yield).

¹H NMR (400 MHz, Chloroform-d) δ=7.75-7.71 (m, 2H), 7.29-7.24 (m, 6H), 4.01 (s, 4H), 3.63 (s, 6H), 3.23 (t, J=6.9 Hz, 2H), 2.84-2.79 (m, 2H), 1.87-1.78 (m, 2H) ppm;

¹³C NMR (101 MHz, Chloroform-d) δ=151.2 (2C), 142.2 (2C), 136.1 (2C), 123.0 (2C), 122.3 (2C), 119.8 (2C), 109.3 (2C), 52.2, 51.3 (2C), 49.4, 29.9 (2C), 26.2 ppm;

IR (NaCl pellets): 3382, 3054, 2946, 2096, 1479, 1333, 1128, 747 cm⁻¹.

HRMS: for C₂₁H₂₅N₈⁺ [M+H]⁺: 389.2206, calc. 389.2202.

### PREPARATION 2

### 3-azido-N, N-bis((1-benzyl-1H-1,2,3-triazol-4-yl)methyl)propan-1-amine

### Stage 1: 3-(di(prop-2-yn-1-yl)amino)propan-1-ol

Propargyl bromide (1.46 ml, 13.09 mmol) is added, at ambient temperature, to a solution of aminopropanol (500 μl, 6.54 mmol) and sodium carbonate (2.08 g, 19.64 mmol) in ethanol (20 ml) and then the reaction mixture is stirred overnight at 50° C. After a return to ambient temperature and evaporation under reduced pressure of the reaction mixture, water is added. The aqueous phase is acidified (pH=2-3) by adding a solution of HCl (2N) and then extracted three times with ethyl acetate, and then the organic phases are pooled, dried over MgSO₄ and concentrated. The expected product is obtained in the form of a yellowish oil (600 mg, 3.97 mmol, 60% yield) and used directly in the next step.

¹H NMR (400 MHz, Chloroform-d) δ=3.77 (t, 2H, J=5.4 Hz), 3.47 (d, 4H, J=2.4 Hz), 2.78 (t, 2H, J=6.1 Hz), 2.24 (t, 2H, J=2.4 Hz), 1.71 (quint, 2H, J=5.8 Hz) ppm;

¹³C NMR (101 MHz, Chloroform-d) δ=78.2 (2C), 79.4 (2C), 63.4 (1C), 52.1 (1C), 42.2 (2C), 28.4 (1C) ppm.

### Stage 2: 3-(bis((1-benzyl-1H-1,2,3-triazol-4-yl)methyl)amino)propan-1-ol

In a round-bottomed flask equipped with a magnetic stirrer, the alkyne (9.8 mmol, 1 eq) and benzyl azide (2 eq) are dissolved in an equimolar H₂O/tert-BuOH mixture. The reaction medium is subjected to constant stirring. Sodium ascorbate (9.8 mmol, 1 eq) and copper sulfate pentahydrate (0.98 mmol, 10 mol %) are successively added. The reaction is subjected to stirring at ambient temperature overnight. The reaction crude is concentrated and then purified on an open silica column.

Purification: EtOAc/MeOH Rf=0.35

Yield: 60%

¹H NMR (CDCl₃, 400 MHz): δ (ppm) 7.57 (s, 2H), 7.29 (m, 6H), 7.20 (m, 4H), 5.45 (s, 4H), 3.70 (s, 4H), 3.62 (t, 2H, J=4.0 Hz), 2.67 (t, 2H, J=8.0 Hz), 1.71 (t, 2H, J=8.0 Hz).

¹³C NMR (CDCl₃, 100 MHz): δ (ppm) 144 (2C), 135 (2C), 129 (2C), 128 (2C), 127 (2C), 123 (2C), 62.8, 53.8 (2C), 52.3 (2C), 47.3, 27.9.

FTIR (neat) v (cm⁻¹)=3428, 1643, 1456, 1345, 1173, 1051.

MS (ES+): m/z=418.8

### Stage 3: 3-azido-N,N-bis((1-benzyl-1H-1,2,3-triazol-4-yl)methyl)propan-1-amine

Triethylamine (27 μl, 0.20 mmol) and mesyl chloride (15 μl, 0.20 mmol) are added to a solution of previously obtained alcohol (82 mg, 0.20 mmol) in anhydrous DMF and then the reaction medium is stirred at ambient temperature under an N₂ atmosphere. After disappearance of the starting alcohol (TLC control), sodium azide (64 mg, 0.98 mmol) is added to the reaction mixture. After 12 h of stirring, the reaction crude is concentrated under vacuum, and directly purified by silica column flash chromatography (95/5 EtOAc/MeOH) to give the expected product in the form of a transparent oil (65 mg, 0.15 mmol). Purification: EtOAc/MeOH Rf=0.38

Yield=75%

¹H NMR (CDCl₃, 400 MHz): δ (ppm) 7.51 (s, 2H), 7.34 (m, 6H), 7.25 (m, 4H), 5.49 (s, 4H) δ 3.70 (s, 4H), 3.27 (t, 2H, J=4 Hz), 2.52 (t, 2H, J=8 Hz), 1.79 (quint, 2H, J=8 Hz).

¹³C NMR (CDCl₃, 100 MHz): δ (ppm) 144 (2C), 134 (2C), 130 (2C), 129 (2C), 128 (2C), 123 (2C), 53.9, 49.1, 47.5, 26.3.

FTIR (neat) v (cm⁻¹)=3428, 1643, 1456, 1345, 1173, 1051

MS (ES+): m/z=443.7

### PREPARATION 3

### Diethyl 5,5′-(4,4′-(((3-azidopropyl)azanediyl)bis(methylene))bis(1H-1,2,3-triazole-4,1-diyl))dipentanoate

### Stage 1: Diethyl 5,5′-(4,4′-(((3-hydroxypropyl)azanediyl)bis(methylene))bis(1H-1,2,3-triazole-4,1-diyl))dipentanoate

Ethyl 5-azidopentanoate (3.264 g, 0.0193 mol), CuSO₄.5H₂O (482 mg, 1.93 mmol) and sodium ascorbate (3.82 g, 0.0193 mol) are added to a solution of 3-(di(prop-2-yn-1-yl)amino)propan-1-ol 2 (2.88 g, 0.0193 mol) in 12 ml of water/tert-BuOH. The reaction is subjected to stirring at ambient temperature for 12 h. The reaction crude is evaporated to dryness and purified on a silica column (EtOAc/MeOH) so as to obtain the expected product (2.46 g, yield 52%).

¹H NMR (CDCl₃, 400 MHz): δ (ppm) 7.96 (s, 2H), 4.43 (t, 4H, J=8.0 Hz), 4.10 (q, 4H, J=8.0 Hz), 3.76 (s, 4H), 3.59 (t, 2H, J=8.0 Hz), 3.35 (s, 1H), 2.58 (t, 2H, J=8.0 Hz), 2.36 (t, 4H, J=8.0 Hz), 1.95 (quint, 4H, J=8.0 Hz), 1.78 (quint, 2H, J=8.0 Hz), 1.59 (quint, 4H, J=8.0 Hz), 1.22 (t, 6H, J=8.0 Hz).

¹³C NMR (CDCl₃, 100 MHz): δ (ppm) 172 (2C), 143 (2C), 123 (2C), 62.5 (2C), 60.1, 52.1 (2C), 49.8 (2C), 47.1, 33.1 (2C), 29.2, 27.9 (2C), 21.5 (2C), 13.9 (2C).

FTIR (neat) v (cm⁻¹)=3399, 3139, 2942, 1730, 1459, 1375, 1330, 1256, 1187, 1129, 1052, 790.

MS (ES+): m/z=494.8

### Stage 2: Diethyl 5,5′-(4,4′-(((3-azidopropyl)azanediyl)bis(methylene))bis(1H-1,2,3-triazole-4,1-diyl))dipentanoate

TEA (0.548 mmol) and MsCl (571 mg, 0.274 mmol) are added to a solution of diethyl 5,5′-(4,4′-(((3-hydroxypropyl)azanediyl)bis(methylene))bis(1H-1,2,3-triazole-4,1-diyl))dipentanoate (246 mg, 0.5 mmol) in anhydrous DMF (10 ml). After 3 h of stirring under an inert atmosphere, NaN₃ (486 mg, 0.41 mmol) is added and the reaction mixture is stirred for 12 h at ambient temperature and under N₂. The reaction crude is concentrated under vacuum and the crude product is purified on a silica column (EtOAc/MeOH) to give the expected pure product (193 mg, 0.37 mmol, 74% yield).

¹H NMR (400 MHz, Methanol-d4) δ=7.97 (s, 2H), 4.43 (t, 4H, J=8 Hz), 4.11 (q, 4H, J=8 Hz), 3.75 (s, 4H), 2.36 (t, 4H, J=8 Hz), 1.95 (quint, 4H, J=8 Hz), 1.78 (quint, 2H, J=8 Hz), 1.59 (quint, 4H, J=8 Hz), 1.22 (t, 6H, J=8 Hz) ppm;

¹³C NMR (101 MHz, Methanol-d4) δ=175 (2C), 146 (2C), 125 (2C), 61.6 (2C), 51.1, 49.8 (2C), 34.4 (2C), 30.7 (2C), 27.7, 23.0 (2C), 14.7 (2C) ppm;

IR (NaCl pellets): 3135, 2941, 2097, 1731, 1458, 1374, 1255, 1185, 1047, 822 cm⁻¹;

MS (ES+): m/z=519.8

### Example 1

### 5,5′-(4,4′-(((3-azidopropyl)azanediyl)bis(methylene))bis(1H-1,2,3-triazole-4,1-diyl))dipentanoic acid

EtOH (3.65 ml) is added to a solution of diethyl 5,5′-(4,4′-(((3-azidopropyl)azanediyl)bis(methylene))bis(1H-1,2,3-triazole-4,1-diyl))dipentanoate (946 mg, 1.825 mmol) of preparation 3 in a solution of NaOH (2M, 292 mg), in order to render the reaction medium homogeneous. The solution is stirred at ambient temperature for 24 h and the ethanol is evaporated to dryness. The aqueous phase is acidified to pH=7 with a solution of HCl (1M) and evaporated. The reaction crude is dissolved in ice-cold MeOH and the salts are filtered twice and the expected product is obtained (843 mg, yield 99%).

¹H NMR (400 MHz, Methanol-d4) δ=8.00 (s, 2H), 4.44 (t, 4H, J=8 Hz), 3.76 (s, 4H), 2.72 (t, 2H, J=8 Hz), 2.23 (t, 4H, J=8 Hz), 1.96 (quint, 4H, J=8 Hz), 1.82 (quint, 2H, J=8 Hz), 1.63 (quint, 4H, J=8 Hz) ppm;

IR (NaCl pellets): 4453, 4197, 3944, 3352, 3055, 2985, 2832, 2522, 2306, 2099, 1571, 1420, 1266, 1222, 1126, 1024, 981, 896, 737, 704 cm⁻¹

The compound of example 1 has two COOH groups allowing functionalization of the chelating azide. A conjugate with a rhodamine was obtained as follows:

### Rhodamine A

### 4-((3-((tert-butoxycarbonyl)amino)propyl)carbamoyl)-2-(6-(dimethylamino)-3-(dimethyliminio)-3H-xanthen-9-yl)benzoate

DIPEA (120 μl; 362.6 μmol) and PyBOP (188.7 mg, 725 μmol) are added to a solution of 6-carboxytetramethylrhodamine (156.1 mg, 362.6 μmol) in DMF. After 10 min, N-Boc-1,3-propanediamine is added (63.18 mg, 362.6 μmol) and the reaction mixture is stirred for 4 h in the dark. After evaporation of the solvent, the reaction crude is purified by HPLC, to give the expected product (40 mg, 68.2 μmol, 18.9% yield).

¹H NMR (400 MHz, Methanol-d4): δ=8.68 (s, 1H), 8.18 (d, J=8.0 Hz, 1H), 7.47 (d, J=8.0 Hz, 1H), 7.21 (d, J=9.5 Hz, 2H), 7.05 (d, J=9.5 Hz, 2H), 6.97 (s, 2H), 3.51 (t, J=7.0 Hz, 2H), 3.32 (s, 12H), 3.19 (t, J=7.0 Hz, 2H), 1.88-1.79 (m, 2H), 1.47 (s, 9H)

MS (IES): [M+H]⁺=587.6

### Rhodamine B

### 4-((3-(5-(4-(((3-azidopropyl)((1-(4-carboxybutyl)-1H-1,2,3-triazol-4-yl)methyl)amino)methyl)-1H-1,2,3-triazol-1-yl)pentanamido)propyl)carbamoyl)-2-(6(dimethylamino)-3-(dimethyliminio)-3H-xanthen-9-yl)benzoate

TFA (200 μl) is added, at ambient temperature, to a solution of rhodamine A (12.6 mg, 21.5 μmol) in distilled CH₂Cl₂ (500 μl), and the reaction mixture is stirred for 3 h in the dark. After evaporation of the solvent, the reaction crude is taken up in DMF, and then DIPEA (16 μl; 96.8 μmol), 5,5′-(4,4′-(((3-azidopropyl)azanediyl)bis(methylene))bis(1H-1,2,3-triazole-4,1-diyl))dipentanoic acid (14.9 mg; 32.3 μmol) and PyBOP (16.8 mg, 32.3 μmol) are added. The reaction mixture is stirred at ambient temperature for 4 h in the dark. After evaporation of the solvent, the reaction crude is purified by HPLC, to give the expected product (1.4 mg, 1.5 mmol, 7% yield).

¹H NMR (400 MHz, Methanol-d4): b=8.74 (s, 1H), 8.21 (d, J=8.0 Hz, 1H), 8.13 (s, 1H), 8.09 (s, 1H), 7.49 (d, J=8.0 Hz, 1H), 7.18 (d, J=9.5 Hz, 2H), 7.06 (d, J=9.5 Hz, 2H), 6.98 (s, 2H), 4.52-4.40 (m, 6H), 4.08-3.99 (m, 4H), 3.54-3.46 (m, 4H), 3.34-3.30 (m, 20H), 2.32-2.20 (m, 4H), 2.01-1.90 (m, 4H), 1.88-1.81 (m, 1H), 1.63-1.55 (m, 1H)

MS (IES): [M+H]⁺=931.6

### PREPARATION 4

### Ethyl 5-(4-(((3-azidopropyl)((1-(tert-butyl)-1H-1,2,3-triazol-4-yl)methyl)amino)methyl)-1H-1,2,3-triazol-1-yl)pentanoate

### Stage 1: ethyl 5-(4-(((3-hydroxypropyl)(prop-2-yn-1-yl)amino)methyl)-1H-1,2,3-triazol-1-yl)pentanoate

CuSO₄.5H₂O (207 mg, 8.7 mmol) and sodium ascorbate (1.72 g, 0.0873 mol) are added to a solution of 3-(di(prop-2-yn-1-yl)amino)propan-1-ol 2 (1.32 g, 0.0873 mol) in 5 ml of water/tert-BuOH, then ethyl 5-azidopentanoate (1.49 g, 0.0873 mol) dissolved in 5 ml of water/tert-BuOH is added over the course of 5 h with a syringe driver. The reaction is subjected to stirring at ambient temperature for 12 h. The reaction crude is evaporated to dryness and purified on a silica column (EtOAc/MeOH, 90/10) so as to obtain the expected product (450 mg, 13.9 mmol, yield 16%).

¹H NMR (MeOD, 400 MHz): δ 7.90 (s, 1H), 4.42 (t, 2H, J=8.0 Hz), 4.11 (q, 2H, J=8.0 Hz), 3.82 (s, 2H), 3.61 (t, 2H, J=8.0 Hz), 3.39 (s, 2H), 2.69-2.65 (m, 3H), 2.36 (t, 2H, J=8.0 Hz), 1.94 (tt, 2H, J=8.0 Hz), 1.73 (tt, 2H, J=8.0, 8.0 Hz), 1.60 (tt, 2H, J=8.0, 8.0 Hz), 1.24 (t, 3H, J=8.0 Hz) ppm.

¹³C NMR (CDCl₃, 100 MHz): δ 172.7, 144.2, 122.4, 77.6, 73.7, 62.9, 60.2, 53.3, 51.9, 49.7, 48.3, 41.7, 33.2, 29.4, 28.2, 28.1, 21.5, 14.0 ppm.

IR (NaCl pellets): v=3291, 2948, 1725, 1462, 1440, 1122, 1064 cm⁻¹.

### Stage 2: Ethyl 5-(4-((((1-(tert-butyl)-1H-1,2,3-triazol-4-yl)methyl)(3-hydroxypropyl)amino)methyl)-1H-1,2,3-triazol-1-yl)pentanoate

2-Azido-2-methylpropane (596 g, 596 μl, 6.024 mmol), CuSO₄.5H₂O (49.94 mg, 0.20 mmol) and sodium ascorbate (397.8 mg, 2.008 mol) are added to a solution of ethyl 5-(4-(((3-hydroxypropyl)(prop-2-yn-1-yl)amino)methyl)-1H-1,2,3-triazol-1-yl)pentanoate (647 mg, 2.008 mmol) in 5 ml of water/tert-BuOH. The reaction is subjected to stirring at ambient temperature for 12 h. Since the reaction is not complete, the same amounts of copper and of ascorbate are added at the same time as two equivalents of 2-azido-2-methylpropane. The reaction crude is evaporated to dryness and purified on a silica column (EtOAc/MeOH) so as to obtain the expected product (425 mg, yield 50%).

¹H NMR (400 MHz, Chloroform-d) δ=7.72 (s, 1H), 7.70 (s, 1H), 4.34 (t, 2H, J=8 Hz), 4.09 (q, 2H, J=8 Hz), 3.77 (s, 2H), 3.75 (s, 2H), 3.73 (t, 2H, J=8 Hz), 2.76 (t, 2H, J=8 Hz), 2.31 (t, 2H, J=8 Hz), 1.81 (quint, 2H, J=8 Hz), 1.71 (quint, 2H, J=8 Hz), 1.64 (s, 9H), 1.21 (t, 3H, J=8 Hz) ppm;

¹³C NMR (101 MHz, Methanol-d4) δ=175, 173, 145, 144, 126, 123, 117, 61.6, 61.1, 51.8, 51.1, 34.4, 30.7, 30.3 (3C), 23.0, 21.0, 14.6 ppm;

IR (NaCl pellets): 3412, 2940, 1729, 1612, 1372, 1197, 1051 cm⁻¹.

### Stage 3: Ethyl 5-(4-(((3-azidopropyl)((1-(tert-butyl)-1H-1,2,3-triazol-4-yl)methyl)amino)methyl)-1H-1,2,3-triazol-1-yl)pentanoate

TEA (198 mg, 1.952 mmol) and MsCl (112 mg, 0.976 mmol) are added to a solution of ethyl 5-(4-((((1-(tert-butyl)-1H-1,2,3-triazol-4-yl)methyl)(3-hydroxypropyl)amino)methyl)-1H-1,2,3-triazol-1-yl)pentanoate (411 mg, 0.976 mmol) in anhydrous DMF (4 ml). After 2 h of stirring under an inert atmosphere, NaN₃ (95.2 mg, 1.464 mmol) is added and the reaction mixture is stirred for 12 h at ambient temperature and under N₂. The reaction crude is concentrated under vacuum and the crude product is purified on a silica column (EtOAc/MeOH) so as to give the expected pure product (130 mg, 30% yield).

¹H NMR (400 MHz, Methanol-d4) δ=8.03 (s, 1H), 7.97 (s, 1H), 4.43 (t, 2H, J=8 Hz), 4.09 (q, 2H, J=8 Hz), 3.76 (s, 2H), 3.75 (s, 2H), 3.35 (t, 2H, J=4 Hz), 2.52 (t, 2H, J=8 Hz), 2.36 (t, 2H, J=8 Hz), 1.95 (quint, 2H, J=8 Hz), 1.81 (quint, 2H, J=8 Hz), 1.68 (s, 9H), 1.60 (quint, 2H, J=4 Hz), 1.21 (t, 3H, J=8 Hz) ppm;

IR (NaCl pellets): 3417, 2940, 2097, 1730, 1459, 1373, 1208, 1047, 827 cm⁻¹

### Example 2

### 5-(4-(((3-azidopropyl)((1-(tert-butyl)-1H-1,2,3-triazol-4-yl)methyl)amino)methyl)-1H-1,2,3-triazol-1-yl)pentanoic acid

EtOH (300 μl) is added to a solution of ethyl 5-(4-(((3-azidopropyl)((1-(tert-butyl)-1H-1,2,3-triazol-4-yl)methyl)amino)methyl)-1H-1,2,3-triazol-1-yl)pentanoate of preparation 4 (94.5 mg, 0.2286 mmol) in a solution of NaOH (2M, 17.73 mg), in order to render the reaction medium homogeneous. The solution is stirred at ambient temperature for 24 h and the ethanol is evaporated to dryness. The aqueous phase is acidified to pH=7 with a solution of HCl (1M) and evaporated. The reaction crude is dissolved in ice-cold MeOH and the salts are filtered twice and the expected product is obtained (96 mg, yield 99%).

¹H NMR (400 MHz, Methanol-d4) δ=8.11 (s, 1H), 8.06 (s, 1H), 4.45 (t, 2H, J=8 Hz), 3.91 (s, 2H), 3.90 (s, 2H), 3.36 (t, 2H, J=4 Hz), 2.66 (t, 2H, J=4 Hz), 2.34 (t, 2H, J=8 Hz), 1.97 (quint, 2H, J=8 Hz), 1.86 (quint, 2H, J=8 Hz), 1.68 (s, 9H), 1.60 (quint, 2H, J=8 Hz) ppm;

¹³C NMR (101 MHz, Methanol-d4) δ=177, 144, 143, 126, 123, 31.1, 51.2, 34.4, 30.8, 30.3 (3C), 27.1, 23.1 ppm;

IR (NaCl pellets): 3408, 2979, 2364, 2099, 1711, 1461, 1209, 1052, 823 cm⁻¹

### PREPARATION 5

### di-tert-butyl(((4,4′-(4,4′-(((3-azidopropyl)azanediyl)-bis(methylene))bis(1H-1,2,3-triazole-4,1-diyl))bis(butanoyl))bis(azanediyl))-bis(propane-3,1-diyl))dicarbamate

### Stage 1: di-tert-butyl(((4,4′-(4,4′-(((3-hydroxypropyl)azanediyl)bis(methylene))-bis(1H-1,2,3-triazole-4,1-diyl))bis(butanoyl))bis(azanediyl))bis(propane-3,1-diyl))dicarbamate

tert-Butyl 3-(4-azidobutanamido)propyl)carbamate (1.38 g, 4.8 mmol), CuSO₄.5H₂O (120 mg, 0.48 mmol) and sodium ascorbate (950 mg, 4.8 mmol) are added to a solution of 3-(di(prop-2-yn-1-yl)amino)propan-1-ol 2 (731 mg, 4.8 mmol) in 10 ml of water/tert-BuOH (1:1). The reaction is subjected to stirring at ambient temperature for 12 h. The reaction crude is evaporated to dryness and purified on a silica column (CH₂Cl₂/MeOH 8:2) so as to obtain the expected product (820 mg, yield 24%).

¹H NMR (400 MHz, Chloroform-d) δ=7.74 (s, 2H), 7.00 (br. s, 2H), 5.14 (br. s, 2H), 4.42 (t, J=6.0 Hz, 4H), 3.86 (s, 4H), 3.68 (t, J=5.0 Hz, 2H), 3.29-3.27 (m, 4H), 3.16-3.13 (m, 4H), 2.93-2.90 (m, 2H), 2.25-2.21 (m, 4H), 2.17-2.14 (m, 4H), 1.84-1.82 (m, 2H), 1.65-1.63 (m, 4H), 1.41 (s, 18H) ppm;

¹³C NMR (101 MHz, Chloroform-d): δ=171.7 (2C), 156.4 (2C), 143.2 (2C), 124.1 (2C), 79.0 (2C), 62.4 (2C), 52.6, 49.3 (2C), 48.0 (2C), 37.3, 36.1, 32.2 (2C), 29.8 (2C), 28.3 (6C), 28.0 (2C), 25.8 (2C) ppm;

MS (IES): [M+H]⁺=722.6.

### Stage 2: di-tert-butyl(((4,4′-(4,4′-(((3-azidopropyl)azanediyl)-bis(methylene))bis(1H-1,2,3-triazole-4,1-diyl))bis(butanoyl))bis(azanediyl))-bis(propane-3,1-diyl))dicarbamate

TEA (296 μl, 2.12 mmol) and MsCl (60 μl, 0.777 mmol) are added to a solution of di-tert-butyl(((4,4′-(4,4′-(((3-hydroxypropyl)azanediyl)bis(methylene))bis(1H-1,2,3-triazole-4,1-diyl))bis(butanoyl))bis(azanediyl))bis(propane-3,1-diyl))dicarbamate (530 mg, 0.706 mmol) from stage 1 in anhydrous DMF (20 ml). After 2 h of stirring under an inert atmosphere, sodium azide (230 mg, 3.53 mmol) is added and the reaction mixture is stirred for 12 h at ambient temperature under N₂. The reaction crude is concentrated under vacuum and the residue is purified on a silica column (EtOAc/MeOH, 80:20) so as to give the expected pure product (270 mg, 0.348 mmol, 49% yield).

¹H NMR (400 MHz, Chloroform-d): δ=7.68 (s, 2H), 6.83 (br. s, 2H), 5.05 (br. s, 2H), 4.42 (t, J=6.5 Hz, 4H), 3.76 (s, 4H), 3.37 (t, J=6.5 Hz, 2H), 3.31-3.29 (m, 4H), 3.16-3.14 (m, 4H), 2.73-2.71 (m, 2H), 2.26-2.24 (m, 4H), 2.19-2.16 (m, 4H), 1.91-1.88 (m, 2H), 1.65-1.63 (m, 4H), 1.42 (s, 18H) ppm;

¹³C NMR (101 MHz, Chloroform-d): δ=171.7 (2C), 156.6 (2C), 148.5 (2C), 124.3 (2C), 79.4 (2C), 51.1, 49.5 (2C), 49.3 (2C), 47.8 (2C), 37.4, 36.2 (2C), 32.6 (2C), 30.2 (2C), 28.5 (6C), 26.5, 26.1 (2C) ppm.

### Example 3

### 4,4′-(4,4′-(((3-azidopropyl)azanediyl)bis(methylene))bis(1H-1,2,3-triazole-4,1-diyl))bis(N-(3-aminopropyl)butanamide)

1 ml of TFA is added to a solution of di-tert-butyl(((4,4′-(4,4′-(((3-azidopropyl)azanediyl)bis(methylene))bis(1H-1,2,3-triazole-4,1-diyl))bis(butanoyl))bis(azanediyl))bis(propane-3,1-diyl))dicarbamate (200 mg, 0.268 mmol) of preparation 5 in 4 ml of CH₂Cl₂. After 2 h of stirring at ambient temperature, the medium is concentrated to give the desired product in the form of a colorless oil (208 mg, 0.268 mmol, quanti.).

¹H NMR (400 MHz, Methanol-d): δ=8.16 (s, 2H), 4.38 (s, 4H), 4.35 (t, J=6.5 Hz, 4H), 3.32 (t, J=6.5 Hz, 2H), 3.14-3.09 (m, 6H), 2.81 (t, J=7.5 Hz, 4H), 2.12-2.04 (m, 10H), 1.71-1.68 (m, 4H), ppm;

¹³C NMR (101 MHz, Methanol-d): δ=175.3 (2C), 137.6 (2C), 128.7 (2C), 51.6, 49.8 (2C), 47.9 (2C), 38.5, 38.4 (2C), 37.1 (2C), 33.4 (2C), 28.7 (2C), 27.3 (2C), 25.0 ppm.

### Experiment 1

### Measurement of the Activity of the Azides with Respect to Azide/Alkyne “Click” Reaction in a Buffered Medium

The azide/alkyne coupling reaction kinetics obtained using azides of the present invention and also the kinetics obtained using copper-chelating compounds and azides of the prior art were studied.

The compounds of the prior art were the following:

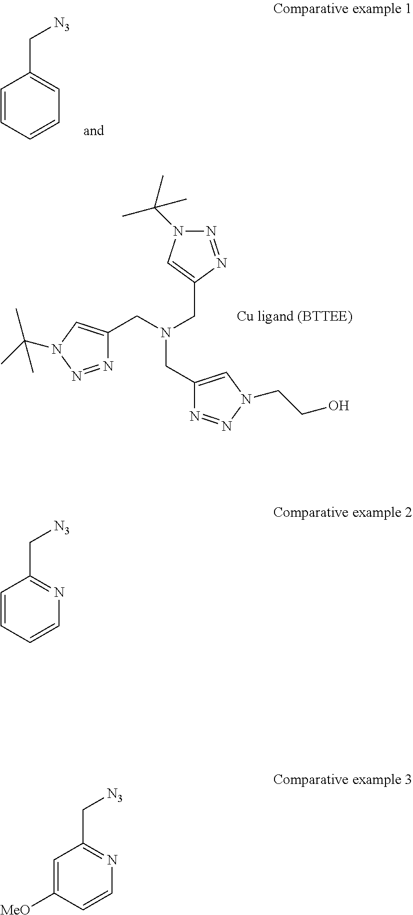 [SMILES: [N-]=[N+]=NCc1ccccc1 ; CC(C)(C)n1cc(CN(Cc2cn(CCO)nn2)Cc2cn(C(C)(C)C)nn2)nn1 ; [N-]=[N+]=NCc1ccccn1 ; COc1ccnc(CN=[N+]=[N-])c1]

The operating conditions were the following:

The kinetics were done under concentration conditions conventional in bioconjugation, i.e. in an aqueous medium at neutral pH and under conditions of low concentration of reagents: azide, alkyne and Cu 17.5 μM, sodium ascorbate 475 μM (25 eq).

The process was more specifically carried out as follows:

The reactivity of the azides of the invention with respect to the azide/alkyne “click” reaction was evaluated by means of a fluorescent test (scheme 1) which makes it possible to establish the kinetic parameters of the reaction and to compare them with the solutions from the literature. This test uses a pro-fluorescent alkyne C1 which forms a highly fluorescent cycloaddition product C2. The synthesis and the fluorescence properties are described in the literature.

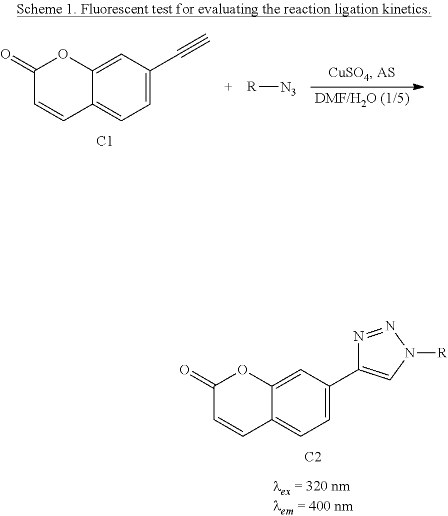

The kinetic studies were carried out under the following conditions:

100 μl of a solution containing 35 μM of alkyne C1 and 35 μm of azide RN₃ dissolved in a DMF/H₂O mixture (50/50) are added to 50 μl of an aqueous solution containing 70 μM of CuSO₄. The reaction is then initiated by adding 50 μl of an aqueous solution containing 1.75 mM of ascorbic acid (AS). The cycloaddition reaction is monitored by measuring, every 5 seconds, for 20 minutes, the fluorescence intensity at 400 nm following excitation at 320 nm. The reaction yield is calculated by means of a calibration range previously established with the cyclo-adduct C2.

The results obtained are the following:

| | Compound | Reaction rate | |
|---|---|---|---|
| | tested | (μM/min) | Ligation yield |
| Comparative example 1 | | 5.2 | 33% |
| Comparative example 2 | | 4.2 | 28% |
| Comparative example 3 | | 3.5 | 35% |
| Example | Preparation 1 | 101.7 | 65% |
| Example | Preparation 2 | 10.8 | 45% |
| Example | Preparation 3 | 16.5 | 67% |
| Example | Example 1 | 12.0 | 74% |

The kinetics are represented in FIG. 4.

The reaction rates and also the ligation yields are, overall, higher for the azides of the present invention in comparison with those of the comparable compounds of the prior art. The azide of preparation 1 for example allows a “click” reaction which is approximately 20 times faster, and the azide of preparation 3 or of example 1 allows yields which are twice as high as those of the best comparable compounds of the prior art.

### Experiment 2

### Ligation Kinetics in a Complex Medium

An experiment was also carried out in a cell culture medium. The process was carried out as follows:

The kinetic studies were carried out under the following conditions:

100 μl of a solution containing 35 μM of alkyne C1 and 35 μM of azide RN₃ dissolved in cell lysate are added to 50 μl of cell lysate containing 1.4 mM of CuSO₄. The reaction is then initiated by adding 50 μl of an aqueous solution containing 1.75 mM of ascorbic acid (AS). The cycloaddition reaction is monitored by measuring, every 5 seconds, for 20 minutes, the fluorescence intensity at 400 nm following excitation at 320 nm. The reaction yield is calculated by means of a calibration range previously established with the cyclo-adduct C2.

The results obtained are represented in FIG. 5, which therefore shows the comparative kinetics, in a complex medium (cell lysate) of formation of the product C2 as a function of the azides used. Conditions: Azide and alkyne 17.5 μM, Cu 350 μM, sodium ascorbate 8.75 mM (500 eq).

The upper curve corresponds to the azide of example 2, the lower curves to comparative examples 1 and 3 and the middle curve to preparation 3.

Examination of the figure shows that the difference in reactivity between the azides of the present invention and the compounds of the prior art is even more pronounced when the “click” reaction is carried out in a complex medium, such as a cell lysate medium.

### Experiment 3

### Ligation Kinetics in Simple and Complex Medium

The F1 and F2 groups of the compounds of the present invention have a double functionality, as will be shown hereinafter. Specifically,

i) they allow coupling of the chelating azide via a functional group located on F1 and/or on F2 and

ii) they modulate the reactivity of the chelating azide with respect to the click reaction.

The reaction kinetics were determined by means of a fluorescent test illustrated by the reaction below, making it possible to establish the kinetic parameters of the reaction and to compare them with the solutions from the literature. This test uses a pro-fluorescent alkyne which forms a highly fluorescent cycloaddition product.

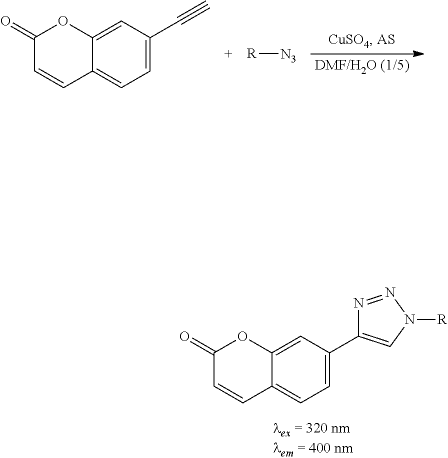

The compound named A14, which is a structural azide similar to those described in the articles by Gilles Gasser et al. “Synthesis, characterization and bioimaging of a fluorescent rhenium-containing PNA bioconjugate”, Dalton Transactions, vol 41, no. 8, 20 Dec. 2011, pp. 2304-2313 and Gasser et al. “Preparation 99m Tc labeling and biodistribution studies of a PNA oligomer containing a new ligand derivate of 2,2-dipicolylamine”, Journal of Inorganic Chemistry, vol 104, no. 11, Jul. 31, 2010, pp. 1133-1140 and also the compounds of examples 1 and 2 of the present invention, respectively named A4 and A20, were tested

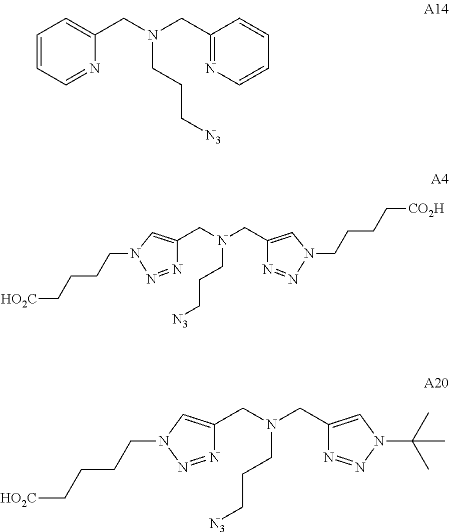 [SMILES: [N-]=[N+]=NCCCN(Cc1ccccn1)Cc1ccccn1 ; [N-]=[N+]=NCCCN(Cc1cn(CCCCC(=O)O)nn1)Cc1cn(CCCCC(=O)O)nn1 ; CC(C)(C)n1cc(CN(CCCN=[N+]=[N-])Cc2cn(CCCCC(=O)O)nn2)nn1]

The operating conditions are the following: the same concentration of 17 μM was used for the alkyne and for the azide. The copper sulfate concentration was 34 μM and the sodium ascorbate (AS) concentration was 850 μM.

The process was carried out, on the one hand, in a 0.1M phosphate buffer at pH 7.4 and, on the other hand, in a cell lysate obtained by means of three cycles of sonication of Jurkat (human myeloma) for 30 seconds. The alkyne, the azide and the copper sulfate are mixed at ambient temperature for 30 minutes and then the kinetics are initiated by adding sodium ascorbate.

The results of the kinetics are shown in FIGS. 6 and 7. As is shown in FIGS. 6 and 7, the compound A14 is inactive under the conditions used, whether in a complex medium such as a cell lysate or a very simple medium such as the phosphate buffer. Only the chelating azides of the invention (A4, A20) make it possible to carry out the click reaction with efficiency. The influence of the F1 and F2 groups on the efficiency of the reaction is also demonstrated in the results illustrated by FIG. 1. A difference in reactivity is already observed between the compounds of examples 1 and 2 in the phosphate buffer medium. This difference becomes more than significant in a complex medium such as that of a cell lysate. Under these conditions, the compound of example 2 is particularly effective.

### Experiment 4

### Ligation in Living Cells

The notable efficiency of the chelating azide compounds of the invention was confirmed by means of experiments carried out on living cells. The results are illustrated in FIG. 8.

The fluorescent compound 1, the structure of which is shown below, was first prepared

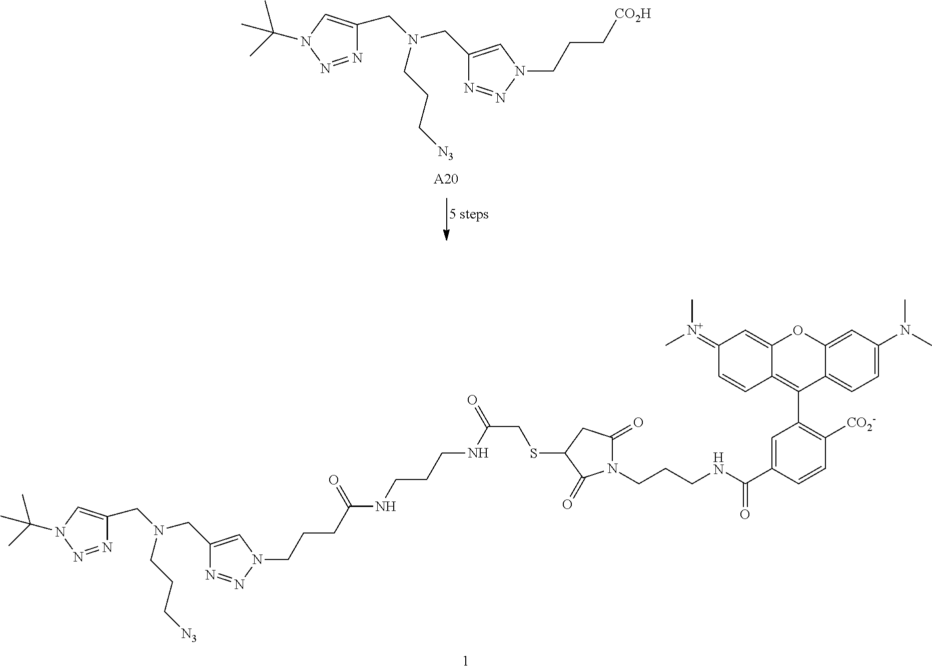

from the compound of example 2 in five steps, using its carboxylic acid function, by proceeding as follows:

### Stage A: tert-butyl(3-(4-(4-(((3-azidopropyl)((1-(tert-butyl)-1H-1,2,3-triazol-4-yl)methyl)amino)methyl)-1H-1,2,3-triazol-1-yl)butanamido)propyl)carbamate

One equivalent of NHS and DCC are added to a solution of compound of example 2 (A20) (213 mg, 0.49 mmol) in 5 ml THF. The reaction is stirred at ambient temperature for 1 h. N-Boc-1,3-propanediamine (0.297 g, 1.7 mmol) dissolved in 5 ml THF is added to the solution, which is stirred overnight at ambient temperature. After evaporation of the solvent, the residue is purified on silica gel so as to obtain 118 mg (0.22 mmol, yield 45%) of the expected product.

¹H NMR (400 MHz, Chloroform-d) δ=7.66 (s, 1H), 7.63 (s, 1H), 6.85 (broad s, 1H), 5.04 (broad s, 1H), 4.42 (t, J=6.0 Hz, 2H), 3.74 (s, 2H), 3.67 (s, 2H), 3.37-3.31 (m, 4H), 3.16 (dt, J=6.0 Hz, J=6.0 Hz, 2H), 2.63 (t, J=6.5 Hz, 2H), 2.27-2.15 (m, 4H), 1.86 (tt, J=6.5 Hz, J=6.5 Hz, 2H), 1.67-1.62 (m, 11H), 1.42 (s, 9H);

¹³C NMR (101 MHz, Chloroform-d) δ=171.7, 156.4, 143.9, 143.6, 123.9, 120.2, 79.2, 77.2, 59.3, 50.5, 49.3, 49.1, 47.8, 47.5, 32.5, 30.1, 30.0, 29.6, 28.4, 26.6, 26.1;

IR (NaCl, cm⁻¹): 3375, 2978, 2940, 2098, 1689, 1652, 1533, 1454, 1368, 1276, 1252, 1170, 1051;

MS (ESI) m/z: 562 [M+H]⁺.

### Stage B: N-(3-aminopropyl)-4-(((3-azidopropyl)((1-(tert-butyl)-1H-1,2,3-triazol-4-yl)methyl)amino)methyl)-1H-1,2,3-triazol-1-yl)butanamide

Tert-butyl(3-(4-(4-(((3-azidopropyl)((1-(tert-butyl)-1H-1,2,3-triazol-4-yl)methyl)amino)methyl)-1H-1,2,3-triazol-1-yl)butanamido)propyl)carbamate (95 mg, 0.17 mmol) is dissolved in 0.5 ml of dichloromethane and then 200 μl of TFA are added. After 2 h of reaction, the solvent is evaporated off and the expected product is obtained in the form of a colorless oil (97.6 mg, 0.17 mmol, yield 100%).

¹H NMR (400 MHz, Methanol-d₄) δ=8.35 (s, 1H), 8.32 (s, 1H), 4.54-4.52 (m, 6H), 3.49 (t, J=6.5 Hz, 2H), 3.29-3.22 (m, 4H), 2.96 (t, J=7.0 Hz, 2H), 2.30-2.21 (m, 4H), 2.18-2.11 (m, 2H), 1.84 (tt, J=7.0 Hz, J=7.0 Hz, 2H), 1.71 (s, 9H);

¹³C NMR (101 MHz, Methanol-d₄) δ=175.2, 137.4, 137.0, 128.5, 125.6, 61.6, 51.5, 49.5, 48.1, 47.9, 38.3, 37.0, 33.2, 30.1, 28.7, 27.1;

IR (NaCl, cm⁻¹): 3405, 2985, 2929, 2105, 1678, 1646, 1553, 1466, 1428, 1374, 1267, 1202, 1131, 1055, 1026, 835, 800, 723, 474;

MS (ESI) m/z: 461 [M+H]⁺.

HRMS (ESI): calculated for C₂₀H₃₇N₁₂O⁺[M+H]⁺: 461.3213. found: 461.3220.

### Stage C: S-(2-((3-(4-(4-(((3-azidopropyl)((1-(tert-butyl)-1H-1,2,3-triazol-4-yl)methyl)amino)methyl)-1H-1,2,3-triazol-1-yl)butanamido)propyl)amino)-2-oxoethyl)ethanethioate

N-(3-Aminopropyl)-4-(((3-azidopropyl)((1-(tert-butyl)-1H-1,2,3-triazol-4-yl)methyl)amino)methyl)-1H-1,2,3-triazol-1-yl)butanamide (71.4 mg, 124 μmol) is dissolved in 1.5 ml of DMF, TEA is added (21 μl, 149 μmol), and the reaction is stirred at ambient temperature for 10 min. 2,5-Dioxopyrrolidin-1-yl-2-(acetylthio)acetate is added and the reaction is stirred for 5 h. The solvent is evaporated off and then the residue is purified on a silica column (CH₂Cl₂/MeOH: 90/10) so as to give the expected product (38 mg, 66 μmol, yield 53%).

¹H NMR (400 MHz, Chloroform-d) δ=7.67 (s, 1H), 7.63 (s, 1H), 6.94 (broad s, 2H), 4.42 (t, J=6.0 Hz, 2H), 3.74 (s, 2H), 3.66 (s, 2H), 3.55 (s, 2H), 3.36 (t, J=6.5 Hz, 2H), 3.32-3.21 (m, 4H), 2.62 (t, J=6.5 Hz, 2H), 2.40 (s, 3H), 2.28-2.13 (m, 4H), 1.86 (tt, J=6.5 Hz, J=6.5 Hz, 2H), 1.66 (s, 9H);

¹³C NMR (101 MHz, Chloroform-d) δ=195.5, 172.0, 168.4, 144.0, 143.6, 124.0, 120.2, 59.4, 50.5, 49.3, 49.1, 47.8, 47.4, 36.4, 36.0, 33.1, 32.5, 30.3, 30.0, 29.4, 26.6, 26.1;

IR (NaCl, cm⁻¹): 3413, 2097, 1646, 1545, 1440, 1372, 1210, 1133, 1049, 960, 489, 459;

MS (ESI) m/z: 577 [M+H]⁺.

### Stage D: 4-((3-(3((2-((3-(4-(4-(((3-azidopropyl)((1-(tert-butyl)-1H-1,2,3-triazol-4-yl)methyl)amino)methyl)-1H-1,2,3-triazol-1-yl)butanamido)propyl)amino)-2-oxoethyl)thio)-2,5-dioxopyrrolidin-1-yl)propyl)carbamoyl)-2-(6-(dimethylamino)-3-(dimethyliminio)-3H-xanthen-9-yl)benzoate

S-(2-((3-(4-(4-(((3-Azidopropyl)((1-(tert-butyl)-1H-1,2,3-triazol-4-yl)methyl)amino)methyl)-1H-1,2,3-triazol-1-yl)butanamido)propyl)amino)-2-oxoethyl)ethanethioate (16.48 mg, 28.6 μmol) is dissolved in 240 μl of EtOH, 1N NaOH is added (95 μl, 95 μmol), and the reaction is stirred at ambient temperature for 3 h. Tetramethylrhodamine (TAMRA)-maleimide (16.19 mg, 28.6 μmol) dissolved in 1.5 ml of EtOH is added and the reaction is stirred for 1 h 30. The solvent is evaporated off and the residue is purified by preparative HPLC, so as to give the desired product (4.7 mg, 4.3 μmol, yield 15%).

¹H NMR (400 MHz, Chloroform-d) δ=8.81 (s, 1H), 8.27 (dd, J=2.0 Hz, J=8.0 Hz, 1H), 8.19-8.15 (m, 1H), 8.14-8.05 (m, 2H), 8.02 (s, 1H), 7.99 (s, 1H), 7.89-7.82 (broad s, 1H), 7.15 (d, J=9.5 Hz, 1H), 7.14 (d, J=9.5 Hz, 1H), 6.81 (dd, J=2.0 Hz, J=9.5 Hz, 2H), 6.72 (d, J=2.0 Hz, 2H), 4.35-4.25 (m, 4H), 4.05-3.97 (m, 5H), 3.91 (d, J=15.0 Hz, 1H), 3.59-3.44 (m, 3H), 3.36-3.30 (m, 2H), 3.29-3.22 (m, 14H), 3.22-3.16 (m, 3H), 2.84 (t, J=7.5 Hz, 2H), 2.57 (dd, J=3.5 Hz, J=19.0 Hz, 1H), 2.15-2.06 (m, 2H), 2.06-1.95 (m, 6H), 1.67 (s, 9H);

¹³C NMR (101 MHz, Methanol-d₄) δ=178.5, 177.0, 174.4, 171.4, 168.5, 161.2, 159.0, 158.9, 137.7, 137.5, 132.2, 131.5, 131.2, 130.7, 115.4, 114.8, 97.4, 50.0, 49.8, 49.6, 49.5, 49.3, 49.1, 41.1, 40.9, 38.5, 38.1, 37.6, 37.7, 36.7, 35.6, 33.5, 30.1, 30.0, 28.4, 27.3, 26.5;

MS (ESI) m/z: 1101 [M+H]⁺.

The copper complex of this fluorescent compound 1 was then prepared as follows:

The fluorescent compound 1 is mixed in the presence of one equivalent of copper sulfate dissolved in water. The mixture is stirred for 1 h at ambient temperature and then 50 equivalents of sodium ascorbate are added, and the reaction is stirred for 30 minutes so as to quantitatively form the copper(I) complex.

HuH-7 cells fixed on 96-well plates, incubated beforehand in the presence of paclitaxel 2 comprising an alkyne function, were treated with the copper complex above.

The cells were treated with 100 μl of a 62.5 nM solution of compound 2 dissolved in 0.1M phosphate buffer, and then incubated at 37° C. for 30 min. The cells are then washed twice by adding 100 μl of 0.1M phosphate buffer and then treated for 4 h with 100 μl of a 100 μM solution of the copper complex of compound 1. The cells are again washed twice by adding 100 μl of 0.1M phosphate buffer and then fixed by treating with 150 μl of a 4% formaldehyde solution. After fixing for 15 min, the cells are washed twice by adding 100 μl of 0.1M phosphate buffer and then incubated with a 62.5 nM solution of Tubulin Tracker® Green 3 for 30 min at 37° C. The cells are again washed with 0.1M phosphate buffer and then observed by fluorescence microscopy.

After cell washing steps, the fluorescence of the rhodamine part of the copper complex of compound 1 was measured. FIG. 8B shows the fluorescence of the cells incubated with the paclitaxel-alkyne 2 and then treated with the chelating azide 1 in the presence of copper. Compound 3, a covalent conjugate between the paclitaxel and the Oregon Green® fluorophore, was used as a control.

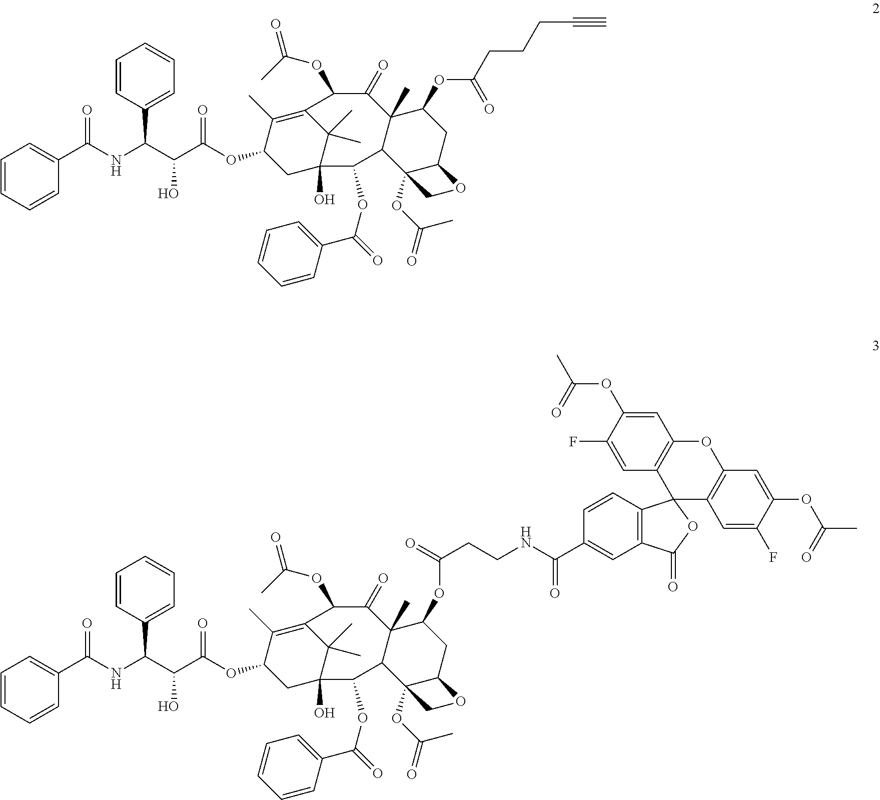 [SMILES: C#CCCCC(=O)O[C@H]1C[C@H]2OC[C@@]2(OC(C)=O)C2[C@H](OC(=O)c3ccccc3)[C@]3(O)C[C@H](OC(=O)[C@H](O)[C@@H](NC(=O)c4ccccc4)c4ccccc4)C(C)=C([C@@H](OC(C)=O)C(=O)[C@@]21C)C3(C)C ; CC(=O)Oc1cc2c(cc1F)C1(OC(=O)c3cc(C(=O)NCCC(=O)O[C@H]4C[C@H]5OC[C@@]5(OC(C)=O)C5[C@H](OC(=O)c6ccccc6)[C@]6(O)C[C@H](OC(=O)[C@H](O)[C@@H](NC(=O)c7ccccc7)c7ccccc7)C(C)=C([C@@H](OC(C)=O)C(=O)[C@@]54C)C6(C)C)ccc31)c1cc(F)c(OC(C)=O)cc1O2]

FIG. 8A illustrates the localization of the cell nucleus by the 4′,6′-diamidino-2-phenylindole reagent. FIG. 8C shows the green fluorescence measured after incubation of compound 3 on the same incubated cells and serves as a control and, finally, FIG. 8D shows the colocalization, by superimposition of the measurements of fluorescence of 1 and 3.

Paclitaxel derivatives are well known to bind with very high affinities to cell tubulin. The experiments illustrated by FIG. 8 show that the azide 1 prepared from the compound of example 2, after having been complexed with copper, is capable of penetrating inside the cells and of coupling, via a “click” reaction, to the paclitaxel-alkyne 2 itself bound to the tubulin as shown in figure B by a red fluorescence.

FIG. 8D, which corresponds to a superimposition of fluorescence images of the red rhodamine linked to compound 1 and of the Oregon Green® linked to compound 3, shows on a color image the colocalization of the molecules (red+green=yellow).

1. An azide of formula I 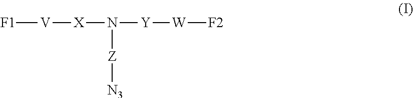
wherein:
V and W represent, independently of one another, a nitrogenous or sulfur-containing heterocycle comprising a copper-complexing heteroatom, said heterocycle being saturated or unsaturated,
X and Y bonded to the central nitrogen represent, independently of one another, a spacer group;
Z represents a spacer group;
F1 and F2 represent, independently of one another, a hydrogen atom, or a tertiary alkylene group having at most 7 carbon atoms, or a functional group which allows the coupling of the azide of formula I to a chemical molecule, to a biomolecule, to a nanoparticle or to a polymer, it being understood that at least one of F1 and F2 represents a functional group.

2. The azide as claimed in claim 1, characterized in that X represents a —(CH₂)ₘ— group, wherein m can take the values 1, 2, 3 or 4.

3. The azide as claimed in claim 1, characterized in that Y represents a —(CH₂)ₘ— group, wherein m can take the values 1, 2, 3 or 4.

4. The azide as claimed in claim 1, characterized in that Z represents an ethylene, propylene or butylene group.

5. The azide as claimed in claim 1, characterized in that V and W represent, independently of one another, a nitrogenous heterocycle.

6. The azide as claimed in claim 1, characterized in that at least one of F1 and F2 represents, independently of the other, a tertiary alkylene group having at most 7 carbon atoms.

7. The azide as claimed in claim 1, characterized in that, when one of F1 and F2 represents a functional group, this group is an isocyanate, isothiocyanate, carboxyl, amino, maleimide or thiol group, indirectly grafted onto the heterocycles.

8. The azide as claimed in claim 1, chosen from
5,5′-(4,4′-(((3-azidopropyl)azanediyl)bis(methylene))bis(1H-1,2,3-triazole-4,1-diyl))dipentanoic acid,
5-(4-(((3-azidopropyl)((1-(tert-butyl)-1H-1,2,3-triazol-4-yl)methyl)amino)methyl)-1H-1,2,3-triazol-1-yl)pentanoic acid and
4,4′-(4,4′-(((3-azidopropyl)azanediyl)bis(methylene))bis(1H-1,2,3-triazol-4,1-diyl))bis(N-(3-aminopropyl)butanamide).

9. A process for preparing an azide as defined in claim 1, wherein X and Y represent, independently of one another, a —(CH₂)ₘ— group, wherein m has the value 1, 2, 3 or 4, characterized in that an amino alcohol of formula II
OH—Z—NH₂ (II)
wherein Z has the meaning already indicated, is reacted with an aldehyde of formula III
GP₁—F₁—V—X′—CHO (III)
wherein F₁ and V have the meaning already indicated, GP₁ represents a group which protects an F1 function and X′ represents a —(CH₂)ₘ₋₁— group wherein m has the value already indicated, so as to obtain a compound of formula IV
GP₁—F₁—V—X′—CH₂—NH—Z—OH (IV)
wherein GP₁, F₁, V, X′ and Z have the meaning already indicated,
which is subjected, with an aldehyde of formula V
GP₂—F₂—W—Y′—CHO (V)
wherein F₂ and W have the meaning already indicated, GP₂ represents a group which protects an F2 function and Y′ represents a —(CH₂)ₘ₋₁— group wherein m has the value already indicated, to a reductive amination so as to obtain a compound of formula VI 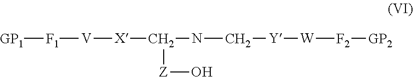
wherein GP₁, F₁, V, X′, Y′, W, F₂, GP₂ and Z have the meaning already indicated, which is reacted with an alkali metal azide of formula VII
EN₃ (VII)
wherein E represents an alkali metal, so as to obtain a compound of formula VIII 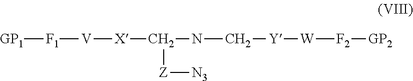
wherein GP₁, F₁, V, X′, Y′, W, F₂, GP₂ and Z have the meaning already indicated,
from which the protective groups are removed so as to obtain the expected compound of formula IA 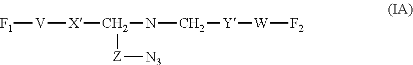
which is isolated if desired.

10. A process for preparing an azide as defined in claim 1, wherein V and W represent a triazole, characterized in that a halogenated alkyne of formula Xa
≡—X—Br (Xa)
wherein X has the meaning already indicated, and Br represents a halogen atom, is reacted with an amino alcohol of formula XI
OH—Z—NH₂ (XI)
wherein Z has the meaning already indicated, so as to obtain a dialkyne of formula XII 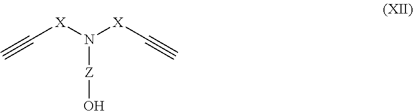
wherein X and Z have the meaning already indicated, which is reacted with an azide of formula XIII
GP₁—F₁—N₃ (XIII)
wherein GP₁ and F₁ have the meaning already indicated, in the presence of a catalyst, so as to obtain by cycloaddition an alkyne of formula XIV, 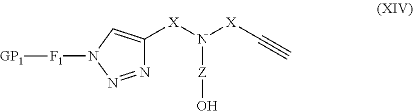
wherein GP₁, F₁, X and Z have the meaning already indicated, which is reacted with an azide of formula XV
GP₂—F₂—N₃ (XV)
wherein GP₂ and F₂ have the meaning already indicated, in the presence of a catalyst, so as to obtain by cycloaddition a compound of formula XVI, 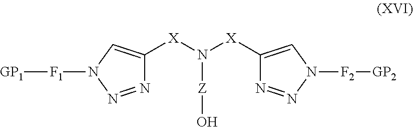
wherein GP₁, GP₂, F₁, F₂, X and Z have the meaning already indicated, which is reacted with an alkali metal azide of formula VII
EN₃ (VII)
wherein E has the meaning already indicated, so as to obtain a compound of formula XVII 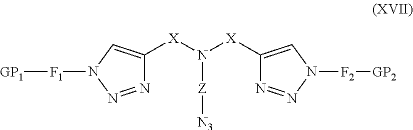
wherein GP₁, GP₂, F₁, F₂, X and Z have the meaning already indicated, from which the protective groups are removed so as to obtain the expected compound of formula IB 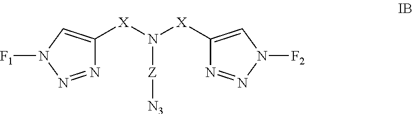
wherein F₁, F₂, X and Z have the meaning already indicated, which is isolated if desired.

11. The use of an azide as defined in claim 1, for the preparation of bioconjugates or of nanoconjugates.

12. The use of an azide as defined in claim 1, for the isolation or identification of a protein target.

13. The use of an azide as defined in claim 1, in imaging.

14. An azide of formula XX 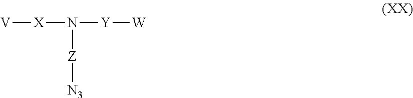
wherein X, Y, V, W and Z have the meaning defined in claim 1.

15. The use of an azide as defined in claim 1, for the isolation or the identification of a protein target within a cell.
